(19) United States
(12) Patent Application Publication
(10) Pub. No.: US 2004/0213748 A1
(43) Pub. Date: Oct. 28, 2004

(54) Antiperspirant formulations

(76) Inventors: Beng Sim Chuah, Selangor (MY); Kevin Ronald Franklin, Bebington,Wirral (GB)

Correspondence Address: UNILEVER, PATENT DEPARTMENT, 45 RIVER ROAD, EDGEWATER, NJ, 07020, US

(21) Appl. No.: 10/483,274

(22) PCT Filed: Jul. 10, 2002
(86) PCT No.: PCT/GB02/03192

(30) Foreign Application Priority Data
Jul. 11, 2001 (GB) 0116946.5

(51) Int. Cl. A61K 7/32

(52) U.S. Cl. 424/65

(57) ABSTRACT

An anhydrous antiperspirant formulation in the form of a soft solid comprising a particulate antiperspirant salt in an amount of from 5 to 30% by weight, an anhydrous carrier fluid in an amount of from 50 to 85% by weight in which at least 70% by weight of the carrier fluid is selected from branched fatty alcohols, aliphatic esters and aromatic esters, and a structurant system for the anhydrous carrier fluid comprising: i) a dibenzylidene alditol in an amount of from 0.05 to 1.5% by weight and ii) a polymeric thickener in an amount of at least 2% by weight.

The present invention relates to antiperspirant formulations and in particular formulations that are anhydrous and in the form of soft solids.

### BACKGROUND AND PRIOR ART

Humans perspire over much of the body, but there are some areas where perspiration is more intense or noticeable after a period of time, such as in the underarm, possibly on account of molecular transformations of excretions from the apocrine, endocrine or sebaceous glands. Antiperspirant formulations are commonly accepted in many societies as a means to obviate or prevent wet patches on human skin or on clothing in contact with the skin. Their application can also reduce body odour generation. The underarm (axilla) is one area where antiperspirant formulations are normally applied.

Antiperspirant formulations are available for application via several different types of dispenser. These include non-contact dispensers such as aerosols or squeeze spray dispensers or contact dispensers such a roll-on or cream or firm stick dispenser. The formulations dispensed include both hydrous or anhydrous compositions. One class of formulation which is favoured by various consumers in some parts of the world comprises anhydrous creams, sometimes called soft solids, in which a particulate antiperspirant active material, commonly an aluminium or aluminium/zirconium astringent salt or complex, is suspended in a water-immiscible carrier fluid which is structured by incorporation of sufficient structurant to enable the formulation to be dispensed under mild pressure through one or more apertures in the dispensing head of a container and remain in place on the dispensing head until applied to the skin, for example the axilla.

Many materials have been contemplated in patent literature or employed in commercial formulations in order to structure carrier fluids. In general, structurants have been separated into distinct classes of structurant, depending on the nature of the carrier fluid. Thus, structurants intended for aqueous or water-miscible carriers such as water, propylene glycol, ethanol or dipropylene glycol or mixtures thereof, have tended to be hydrophylic, whereas structurants intended for water-immiscible carriers such as silicone oils have themselves been water-immiscible. Structurants for the hydrophilic carriers have commonly comprised dibenzylidene alditols, especially dibenzylidene sorbitol, or derivatives of such benzylidene compounds, whereas structurants for water-immiscible carriers have often included various classes of waxes, or organic thickeners, or fibre-forming structurants. The selected structurants are normally considered to dissolve in or at least form a miscible phase with the respective carrier fluids at elevated temperatures, i.e. at a temperature in the region of the melting point of the structurant or higher.

Various anhydrous soft solid antiperspirant formulations containing combinations of organic thickener and other structurants have been disclosed in unpublished International Patent Applications No PCT/EP 01/00186 and PCT/EP 01/00574. The formulations or formulation/package combinations disclosed therein demonstrated many highly desirable properties, such as low or indeed very low visible deposits even after a period of exposure to the atmosphere (leave-on). Some of them appear opaque and others can be translucent when dispensed through narrow apertures. However, the research team investigating soft solids has continued its research and has found that alternative or improved formulations can be obtained by variations in the structurant system not disclosed in either of those patent applications.

It is an object of the present invention to identify alternative or improved soft solid anhydrous formulations containing alternative structurant systems.

### SUMMARY OF THE PRESENT INVENTION

According to a first aspect of the present invention, there is provided an anhydrous antiperspirant formulation in the form of a soft solid comprising:

a particulate antiperspirant salt in an amount of from 5 to 30% by weight; an anhydrous carrier fluid in an amount of from 50 to 85% by weight in which at least 70% by weight of the carrier fluid is selected from branched fatty alcohols, aliphatic esters and aromatic esters, and a structurant system for the anhydrous carrier fluid comprising:

i) a dibenzylidene alditol in an amount of from 0.05 to 1.5% by weight and

ii) a polymeric thickener in an amount of at least 2% by weight

Surprisingly, the instant invention employs a structurant which hitherto has been contemplated for hydrophylic carriers in a water-immiscible carrier fluid. By the incorporation of a dibenzylidene alditol in the selected carrier fluid, it is possible to improve the structurant system by comparison with similar structurant systems in which a dibenzylidene alditol is absent. This can manifest itself in a variety of possible ways. One benefit comprises increasing the thermal stability of the formulation. A further benefit comprises increasing the optical clarity of the formulation and a third benefit comprises reducing the amount of structurant system employed. The balance between the various benefits can be adjusted by variations to the structurant system within the parameters contemplated above and by variations to the remainder of the formulation.

Herein, a soft solid indicates that the formulation is capable of being extruded through a narrow aperture (1 mm diameter) in a dispenser head under application of low pressure, such as 2×10 Pa (about 3 psi) and has a hardness of from 3×10⁻³ N/mm² to 5×10⁻¹ N/mm² as measured by a sphere indentation technique. In many desirable formulations, it has a hardness of from 5×10⁻³ N/mm² to 10⁻¹ N/mm².

Anhydrous herein indicates in the context of a cream composition that the composition does not comprise a liquid aqueous phase.

In a related second aspect of the present invention, there is provided a process for making an anhydrous antiperspirant formulation comprising the steps of:

i) introducing into a mixing vessel a water-immiscible carrier fluid in an amount of from 50 to 85% by weight in which at least 70% by weight of the carrier fluid is selected from branched fatty alcohols, aliphatic esters and aromatic esters;

ii) introducing into the vessel structurant system comprising a dibenzylidene alditol in an amount of at from 0.05 to 1.5% by weight and a polymeric thickener in an amount of least 2% by weight;

iii) heating the structurant system until it melts or is miscible with the carrier fluid;

iv) introducing into the carrier fluid or mixture of carrier fluid and structurant a particulate aluminium/zirconium astringent salt in an amount of from 5 to 30% by weight at a temperature above the normal solidification temperature of the formulation and;

v) introducing the composition into a dispenser whilst the composition is fluid.

In a third and related aspect of the present invention there is provided a cosmetic method for controlling or reducing perspiration in which a composition according to the first aspect is applied topically to human skin.

The present invention is particularly applicable to the manufacture and use of antiperspirant formulations for use in the axilla region of the body.

### DETAILED DESCRIPTION AND PREFERRED EMBODIMENTS

The present invention relates to anhydrous soft solid antiperspirant formulations that are structured using a combination of a dibenzylidene alditol and a polymeric thickener.

The carrier fluid in which the particulate antiperspirant active is suspended comprises at least 70% by weight of selected liquid carriers, namely fatty alcohols and aliphatic or aromatic esters. Fluid in the context of the carrier herein indicates that the alcohol or ester has a melting point that is not higher then ambient temperature, which for convenience is taken to mean not higher than 25° C.

One class of suitable carrier comprises a fatty alcohol, which normally is a branched chain monohydric alcohol which contains from 12 to 24 carbons in total. The carbon length of the branch or branches is commonly from 1 to 10 carbon atoms, such as methyl, ethyl, propyl or octyl. Suitable examples include isocetyl alcohol, isostearyl alcohol and octyl dodecanol.

A second class of suitable carriers comprises aliphatic esters. Suitable aliphatic esters contain at least one long chain alkyl group, such as esters derived from C₁ to C₂₀ alkanols esterified with a C₈ to C₂₋₂ alkanoic acid or C₆ to C₁₀ alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate.

A third class of suitable carrier comprises aromatic esters. Many of the aromatic esters are benzoate esters, others are naphthylate esters and still others are salicylate esters.

Amongst the class of benzoate esters, it is desirable to mention alkyl benzoate, alkylene dibenzoate, alkoxylated alkyl benzoate or a polyalkylene oxide dibenzoate, or a mixture of two or more sub-classes thereof. The alkyl group often contains at least 10 carbons, in many instances up to 25 carbons. It is often linear, but can alternatively be branched.

Especially desirable alkyl groups are found in the range of from 12 to 20 carbons and include dodecyl (lauryl) terdecyl, tetradecyl (myristyl), pentadecy, hexadecyl (palmityl), octadecyl (stearyl) 2-methyl-heptadecyl (iso-stearyl) and octyldodecyl groups. A mixture of two or more of the alkyl groups can be employed, such as a mixture of C₁₂-C₁₅ alkyl groups. The term alkylated herein includes alkylene groups and the latter are terminated at each end with a benzoate group. The alkylene group often contains from 2 to 6 carbons and can be linear or branched, a suitable example of linear being propylene.

In the alkoxylated alkyl benzoates contemplated herein, the alkyl group is terminated by an alkoxy group, which can be monomeric containing for example up to 6 carbons or polymeric such as polyethylene oxide or preferably polypropylene oxide, which conveniently comprises up to 30 units and often from 5 to 20 units. In such compounds, the alkyl group can be selected from the previously identified alkyl groups. Alternatively, the benzoate compound can comprise a polyethylene oxide or polypropylene oxide moiety, or preferably a block copolymer of ethylene oxide and propylene oxide, terminated at each end by a benzoate group. Mixtures of two or more of the benzoate sub-classes of compounds can be employed. Several preferred benzoate compounds are available from Finetex under their trade name Finsolv.

Suitable naphthylate and salicylate esters comprise alkylated naphthylate or salicylate, alkylated being as described above for benzoate esters.

Such carriers, or a mixture of two or more of them, such as an alcohol and an ester, be it aliphatic and/or aromatic, or a mixture of aliphatic and aromatic esters, comprise a proportion of at least 70% by weight of the carrier fluid. Preferably, the proportion is at least 80% and more preferably at least 90%. In some instances the carrier fluid, excluding any miscible fragrance material, is 100% of said alcohol and/or aliphatic and/or aromatic esters. The weight ratio of the alcohol to the aliphatic ester and aromatic ester is at the discretion of the formulator. In many desirable formulations, the aromatic ester represents at least half the total weight of the carrier. In some of such desirable formulations and other formulations the weight ratio of aromatic ester to alcohol is from 1:1 to 10:1.

The balance of the carrier fluid comprises co-carriers which are miscible fluids with the alcohol and/or ester or mixture thereof. Such co-carriers can comprise hydrocarbons, polyalkylene oxide alkyl ethers and silicone fluids.

The hydrocarbons are commonly aliphatic such as mineral oils or hydrogenated polyisobutene, often selected to exhibit a low viscosity. Further examples of liquid hydrocarbons are polydecene and paraffins and isoparaffins of at least 10 carbon atoms. Although polyisobutene and polydecene are polymeric in nature, they are mobile liquids at room temperature of 20° C. and do not cause thickening of other hydrophobic oils. Accordingly they are regarded herein as co-carrier and not as thickener.

The polyalkylene oxide alkyl ethers are ethers derived from a polyglycol moiety and a fatty alcohol moiety. The glycol is often propylene glycol and number of glycol units is often from 3 to 20. The alkyl moiety often comprises from 4 to 15 carbons. Examples of such ethers include myristyl ether derivatives e.g. PPG-3 myristyl ether or lower alkyl ethers of polypropylene glycols such as PPG-14 to PPG-18 butyl ethers e.g. an ether having CFTA name of PPG-14 butyl ether which is commercially available from Amercol under the trade name Fluid AP.

The silicone oils which can be contemplated for incorporation herein can be either volatile or non-volatile oils. Volatile oils can comprise either linear or cyclomethicones containing from 4 to 6 silicon units. Suitable examples include DC245 and DC345, both of which are available from Dow Corning Inc. Non-volatile oils can comprise dimethicones or linear silicone oils which contain a high proportion of phenyl substituents. Suitable examples of non-volatile silicone oils include members of the DC200 series and DC704. However, it is preferable for the proportion of silicone oils in the carrier fluid mixture to be not more than 10%, particularly not more than 5% and most desirable be absent.

One further co-carrier fluid which can be incorporated particularly for its emolliency properties comprises a liquid humectant, and in particular glycerol. Such a fluid can be incorporated in an amount which is miscible with the carrier fluid mixture. A suitable proportion for such an humectant is from 0 to 6 parts by weight of the carrier mixture.

The carrier fluid represents from 55 to 75% by weight of many formulations according to the present invention. Preferably, the formulation contains at least 50% by weight aromatic esters.

The structurant system in the invention formulations comprises a dibenzylidene alditol and a thickening polymer. An optional additional member of the system can comprise a selected co-structurant. A second optional member of the system, whether or not the co-structurant is employed, comprises an alditol dissolution moderator.

The dibenzylidene alditol is conveniently a dibenzylidene sorbitol or xylitol which satisfies the formula below.

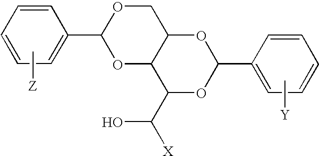

in which X represents CH₂OH or H, and Y and Z represent either ortho, meta or para substituents around the benzene nucleus, which may be the same as each other or different and may be H or F, Cl, Br, C₁-C₃ alkyl or C1-C3 alkoxy substituent.

Preferably, X represents CH₂OH. Alternatively or additionally Y and/or Z represents H. Particularly preferably, Y and Z are the same.

In many desirable formulations according to the present invention, the dibenzylidene alditol comprises at least 0.1% by weight, and in at least some preferred compositions 0.2% by weight or more. In practice, it is often convenient to select the dibenzylidene alditol in an amount of not more than 1% by weight, and in many preferred compositions in an amount of up to 0.5% by weight. The balance of advantages from employing alditol in the invention suspension formulations can be achieved very cost effectively in the range of 0.2 to 0.5%.

The dibenzylidene alditol can, if desired, be employed in conjunction with a gelling temperature moderator. An especially suitable example of such a moderator is propylene carbonate. Conveniently, the moderator can be employed at a weight ratio to the alditol in the range of up to 20:1 and, when employed, often in the range of from 8:1 to 14:1. A convenient weight of moderator in the composition is often from 1 to 4%.

A material which is suitable for use herein as an organic polymeric thickener will generally possess the following characteristics:—

i) it will contain residues of at least 5 (possibly many more than 7) monomer units bonded together into a polymer chain;

ii) it should dissolve on heating in water-immiscible liquids, and specifically it must have a solubility of at least 1.5% by weight in the heated water-immiscible liquid of the continuous phase;

iii) after heating to dissolve and cooling to 20° C., it will increase the viscosity of the water-immiscible liquid of the continuous phase, in the absence of other structurant, when dissolved therein at the same concentration as in the formulation of the invention.

Preferably, under these conditions, it will bring about a viscosity increase of at least 100 mPa.sec, better at least 250 mpa.sec when viscosity is measured with a Brookfield viscometer using a T-bar spindle at 10 rpm at 20° C. The choice of a type B, type C or type D T-bar spindle will depend on the viscosity of the system being measured.

Provided the spindle is appropriate to provide a viscosity measurement it will enable determination of an increase in viscosity brought about by the polymer.

An additional or alternative characterisation of a suitable polymer is that it can thicken the water-immiscible liquid to a viscosity of at least 10,000 mPa.sec, measured in the same way, when incorporated in the water-immiscible liquid at 15% by weight, in the absence of the other structurant.

The polymer will generally be solid at 20° C.

One category of polymer which has been found suitable is a polysaccharide esterified with monocarboxylic acid containing at least 4 carbon atoms.

Preferred in this category is a dextrin fatty acid ester having the formula:—

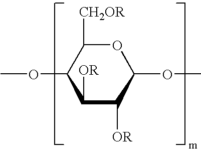

wherein each R group, individually, is a hydrogen atom or an acyl group having up to 22 carbon atoms, provided that at least one R group per glucose unit is an acyl group of at least 4 carbon atoms, and m has an average value from 5, 10 or 20 up to 50 or even up to 150, more preferably from 20 to 30. The dextrin fatty acid ester can be a partial ester, i.e. at least one R group is hydrogen; or the dextrin can be completely esterified, i.e., all R groups are acyl, such as a C₄-C₂₂ acyl group. The acyl groups may be the same or similar, and preferably they are straight chain acyl groups with chain lengths of 8 to 22 carbon atoms, e.g. in a range from 12 or 14 carbon atoms to 18 or 20 carbon atoms. Branched acyl groups may be included, possibly as in a mixture of C₆ to C₂₂ linear acyl groups. Shorter acyl groups may form part of a mixture, for example C₄ to C₈ acyl groups may be mixed with C₁₂ to C₂₂ linear acyl groups. In preferred embodiments, wherein the R group is a C₈-C₂₂ acyl group the degree of substitution is at least 2 (i.e., at least two R groups are C₈-C₂₂ acyl groups).

The C₈-C₂₂ fatty acids that are reacted with the starch hydrolyzate can be saturated or unsaturated acids, and include, for example, capric acid, pelargonic acid, caprylic acid, undecylic acid, undecylenic acid, lauric acid, myristic acid, pentadecylic acid, palmitic acid, heptadecylic acid, stearic acid, nonadecanoic acid, arachidic acid, oleic acid, linoleic acid, linolenic acid, similar acids, and mixtures thereof. These dextrin fatty acid esters are disclosed in Mori et al U.S. Pat. No. 4,780,145, incorporated herein by reference, and some of them are available under the trade name RHEOPEARL from Chiba Flour Milling Co. Ltd., Japan. An example of a dextrin fatty acid ester is dextrin palmitate, available commercially as RHEOPEARL KL and RHEOPEARL FL, for example, from Chiba Flour Milling Co., Ltd. Other examples of esters of C₈-C₂₂ carboxylic acids are dextrin behenate, dextrin laurate, dextrin myristate, dextrin stearate, and mixtures thereof. A second category of polymer which can be used as a thickener comprises polyamides as discussed in U.S. Pat. No. 5,500,209. Such polyamides may be derived from organic diamines containing 2 to 12, preferably 2 to 8 carbon atoms, condensed with di- or poly carboxylic acids containing 4 to 20 carbon atoms per carboxylic acid group. Some monocarboxylic acid may be included in the reaction mixture to control polymer chain length. The dicarboxylic acids may be obtained by thermal polymerisation of unsaturated monocarboxylic acids.

Such polyamides are available from Henkel under their trade name VERSAMID. An example is VERSAMID 950 from hexamethylene diamine and adipic acid.

A further category of polymer which has been found useful is the block copolymers of styrene with ethylene, propylene and/or butylene available from Shell under their trade name KRATON of which some suitable grades are available as KRATON G. Such block copolymers can be either diblock copolymers or triblock copolymers. In the triblock copolymers (sometimes referred to by the abbreviation SEBS) the copolymer comprises a central polyalkylene block terminated at each end by polystyrene blocks. The polymer may be incorporated as such or in the form of a preformed gel of a suitable carrier fluid such as an hydrocarbon oil, eg materials available under the tradename Transgel. Any carrier fluid introduced with the polymer is deemed to form part of the total carrier fluids present in the instant formulations.

Preferred in this category is styrene ethylene/butylene styrene linear block copolymers e.g. that available as KRATON G 1726X.

Another suitable type of polymer is polymers of alpha methylstyrene and styrene available from Hercules under the trade name KRISTALEX. One suitable grade is KRISTALEX F85, with mean molecular weight of approximately 1200.

A further class of polymers found to be suitable for use with a second structurant comprises polyethylene having a molecular weight of from 500, sometimes 2000, to 8000, such as materials available from Quantum USI under the trade name MN 714.

A still further class of polymers found to be suitable comprises co-polymers of vinyl pyrrolidone with polyethylene containing at least 25 methylene units. A particularly suitable polymer comprises triacontanyl polyvinylpyrrolidone, such as that available from International Speciality Products under the trade name Antaron WP-660.

Yet another polymer found to be suitable although less preferred is alkyl substituted galactomannan available from Hercules under their trade name N-HANCE AG.

The thickening ability of polymers varies from one to another, which will affect the amount that is required to attain a desired hardness of the resultant formulation. Normally, the amount of thickening polymer or mixture of polymers is not more than 15% by weight of the composition. Advantageously, the amount will often lie in a range from 2% or 3% by weight of the composition up to 6 or 7%, though a higher amount can be contemplated if desired such as to 10% or 12%.

Beneficially, the combined weight of alditol and polymeric thickener is in many highly desirable formulations as little as from 2 to 6% by weight of the formulation. The weight ratio of thickener to alditol is desirably selected in the range of from 5:1 to 50:1, particularly up to 30:1 and in a number of preferred embodiments is from 8:1 to 20:1.

The structurant system employed herein can comprise a co-structurant. This co-structurant is preferably a fibre-forming structurant and especially a fibre-forming structurant which is selected from the following classes:—

Hydroxystearic acids and derivatives thereof;

structurants which include an amido group;

combinations of a sterol and a sterol ester;

structurants satisfying general formula T1 hereinbelow.

Herein, the term fibre forming structurant indicates a material which forms in a water-immiscible fluid a network of fibres upon cooling to below its crystallisation or gelling temperature.

The co-structurant is preferably present in an amount of up to 2% by weight, and particularly in an amount of up to 1% by weight. It is often present in an amount of at least 0.1% and especially in an amount of at least 0.3%. A number of very desirable formulations herein employ the co-structurant in the range of from 0.4 to 0.6% by weight. The weight ratio of thickener to co-structurant is in many suitable formulations which employ a co-structurant selected in the range of from 3:1 to 12:1.

Within the class of hydroxystearic acids and derivatives thereof, one especially suitable material comprises 12-hydroxystearic acid. Suitable derivatives of 12-hydroxystearic acid include its methyl, ethyl, stearyl or benzyl esters or isopropylamide, butylamide, benzylamide, phenylamide or cyclohexylamide derivatives.

An especially desirable class of co-structurant comprises N-acyl amino acid amides and esters which are known to structure hydrophobic liquids. We have established that they do so by forming fibrous networks. They are described in U.S. Pat. No. 3,969,087, including acylation products of aspartic acid, glutamic acid, glutamine, glycine, sarcosine, α-analine, β analine, α-aminobutyric acid, valine, norvaline, leucine, isoleucine, norleucine, phenylglycine, phenylalanine, serine, threonine, cysteine, methionine, N-acylornithine, γ-aminovaleric acid and ω-aminocaproic acid. The N-acyl moiety contains at least one C5 to C30 alkyl substituent, such as lauroyl, myristoyl, palmitoyl or capryloyl. N-Lauroyl-L-glutamic acid di-n-butylamide is commercially available from Ajinomoto under their designation GP-1.

Further materials which are suitable for use as an amido-containing co-structurant herein are the amide derivatives of di and tribasic carboxylic acids set forth in WO 98/27954. The aliphatic moiety in the amide group is commonly selected in the range of from C1 to C22 alkyl, alkenyl, alkoxy, alkyl ester or alkyl ether, most preferably the alkyl group. Preferred amide materials comprise alkylamides of citric acid, tricarallyic acid, anconitic acid, nitrilotriacetic acid, alkyl succinic acid and alkenylsuccinic acid, and notably alkyl N,N′dialkyl succinamides. In some desirable co-structurants, the N alkyl group is dodecyl and the dialkyl groups are butyl or hexyl.

Further suitable amido-containing co-structurant compounds herein have a general formula A1:

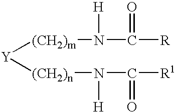

in which Y represents a cyclohexane ring bearing the two substituents indicated above in 1, 2 or 1,3 spacial relationship, m and n and each independently 0 or 1 and R and R¹ may be different or preferably the same and each represents an alkyl group containing from 5 to 27 carbons, preferably 11 to 17 carbons. The alkyl groups are desirably linear. A method for the manufacture of representative amido materials within A1 is described in Example 1 of GB patent application no 0019231.0, which method is hereby incorporated by reference and which can employed, mutatis mutandis, for other A1 amido materials.

A further class of suitable co-structurant is a combination of a sterol and a sterol ester. In its preferred form the sterol satisfies either of the two formulae:

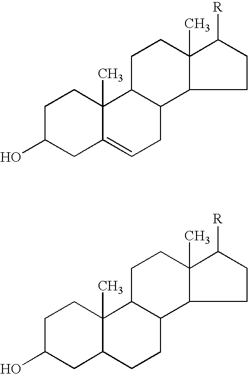

in which R represents an aliphatic, cycloaliphatic or aromatic group, and preferably a linear or branched aliphatic saturated or unsaturated hydrocarbon group. R desirably contains from 1 to 20 carbons and preferably from 4 to 14 carbons.

It is particularly suitable to employ β-sitosterol or campesterol or cholesterol, or a hydrogenated derivative thereof, such as dihydrocholesterol, or a mixture of two or more of them. An especially preferred sterol is β-sitosterol.

The preferred sterol ester is oryzanol, sometimes referred to as γ oryzanol which contains material satisfying the following formula:

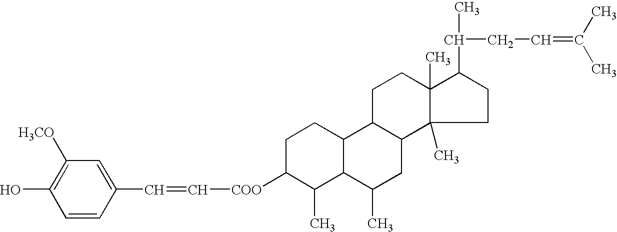

The sterol and sterol ester are used in a mole ratio that is normally selected in the range of from 10:1 to 1:10, especially from 6:1 to 1:4 and preferably in the range of from 3:1 to 1:2. Employment of the two system constituents within such a mole ratio range, and especially within the preferred range facilitates the co-stacking of the constituents and consequently facilitates the formation of a network that is readily able to structure the formulation.

A further suitable co-structurant is in accordance with the following general formula (TI):

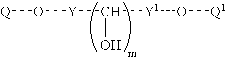

in which Y and Y¹ each represents a methylene or carbonyl group, m is 1 or 2 and Q and Q1 each represent an aralkylene group.

It is preferred that m is 2 so that the structurant compounds comply with a general formula (T2):

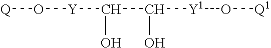

The groups Y and Y¹ will usually be identical, i.e. both methylene or both carbonyl. The groups Q and Q¹ may not be the same but often will be identical to each other.

If m is 2 and Y and Y¹ are methylene groups, the compound is a derivative of threitol, which is 1,2,3,4-tetrahydroxybutane, while if m is 2 and Y and Y¹ are carbonyl groups, the compound is a diester of tartartic acid, which is 2,3-dihydroxybutane-1,4-dioic acid.

It is preferred that each group Q and Q¹ contains an aromatic nucleus which may be phenyl or, less preferably, some other aromatic group. Thus Q and Q¹ may be groups of the formula

Ar—(CH₂)ⁿ—

where Ar denotes an aromatic nucleus, notably phenyl or substituted phenyl and n is from 0 to 10.

The aromatic nucleus (Ar) is preferably unsubstituted or substituted with one or more substituents selected from alkyl, alkyloxy, hydroxy, halogen or nitro.

One substituent may be an alkyl or alkyloxy group with a long alkyl chain. Thus, a formula for preferred structurants of this invention can be given as (T3):

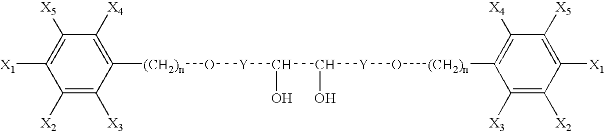

where n=0 to 10, preferably 0 to 3, more preferably 1, 2 or 3;

Y≡CH₂— or >C═O

X₁=H, Cl, Br, F, OH, NO₂, O—R, or R, where R is an aliphatic hydrocarbon chain with 1 to 18 carbon atoms.

X₂ to X₅ are each independently H, Cl, Br, F, OH, NO₂, OCH₃, or CH₃.

In these formulae above, the central carbon atoms that bear hydroxy groups are chiral centres. Thus, if m=2, Y and Y¹ are the same and Q and Q¹ are the same, the compounds will exist as R,R and S,S optically active forms as well as an optically inactive R,S form.

These compounds may be used as their optically active R,R or S,S forms or as a mixture of the two—which may be a racemic mixture.

Compounds within the general formula (TI) above are available commercially. Also, syntheses of these compounds have been given in scientific literature where the compounds were being used as intermediates for purposes not related to the present invention. Thus syntheses of threitol derivatives can be found in:

Kataky et al, J. Chem Soc Perkin Trans vol 2 page 321 [1990], Tamoto et al, Tetrahedron Vol 40 page 4617 [1984], and Curtis et al, J. C. S. Perkin I Vol 15 page 1756 [1977]. Preparations of tartrate esters are found in Hu et al J. Am. Chem. Soc. Vol 118, 4550 [1996] and Bishop et al J. Org Chem Vol 56 5079 [1991].

Antiperspirant actives suitable for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula Al₂(OH)ₓQ_{y}.wH₂O in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever NV et al), the contents of which specification is incorporated herein by reference.

Zirconium actives can usually be represented by the empirical general formula: ZrO(OH)²ⁿ_{-nz}B_{z}.wH₂O in which z is a variable in the range of from 0.9 to 2.0 so that the value 2n−nz is zero or positive, n is the valency of B, and B is selected from the group consisting of chloride, other halide, sulphamate, sulphate and mixtures thereof. Possible hydration to a variable extent is represented by wH₂O. Preferable is that B represents chloride and the variable z lies in the range from 1.5 to 1.87. In practice, such zirconium salts are usually not employed by themselves, but as a component of a combined aluminium and zirconium-based antiperspirant.

The above aluminium and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

It is highly desirable to employ complexes of a combination of aluminium halohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis.

Some antiperspirant actives are produced in the form of dense particles, that is to say are free from voids, or hollow particles that have been milled. It is highly desirable to employ such particles in the context of forming formulations that are translucent when extruded through a narrow aperture and they can also be used in opaque formulations. Other antiperspirant actives that are produced and remain in the form of hollow spheres are particularly suitable for opaque formulations.

Other particulate actives which may be utilised include astringent titanium salts, for example those described in GB 2299506A.

The proportion of solid antiperspirant salt in a composition normally includes the weight of any water of hydration and any complexing agent that may also be present in the solid active. Such hydrated water does not render the formulation hydrous.

The particle size of the antiperspirant salts often falls within the range of 0.1 to 200 μm with a mean particle size often from 3 to 20 μm. Both larger and smaller mean particle sizes can also be contemplated such as from 20 to 50 μm or 0.1 to 3 μm.

A significant factor in determining whether a formulation is translucent or opaque is the difference between the refractive index of the carrier mixture and that of the suspended antiperspirant salt. The mismatch in refractive index can be controlled by a number of treatments to the antiperspirant active and by suitable selection of the carrier fluids. The treatments include milling of antiperspirant actives, thereby removing a void which has a further and different refractive index. A second treatment comprises increasing the extent of hydration of the antiperspirant active. Hydration lowers its refractive index. The refractive index of the carrier fluid is increased by increasing the proportion of aromatic esters relative to aliphatic esters and aliphatic alcohol. It can also be increased by employing a fraction of high refractive index silicone oils, such as the non-volatile arylalkylsilicone oils mentioned previously herein.

Some translucency on extrusion through narrow apertures can be obtained even when the refractive index of the said two constituents does not match exactly, that is to say has a mismatch of greater than 0.003 units, but up to about 0.08 units. In a number of formulations herein, the mismatch is in the region of 0.03 to 0.08 units. Generally, the formulations have limited translucency verging on opaqueness when the mismatch is in the region of 0.08 to 0.1 units and at above 0.1 units is commonly recognised as opaque. It is desirable, none the less, that even for opaque formulations that the mismatch be minimised within the range of not more than 0.11 units.

Other Constituents

Optional ingredients in compositions of this invention can include deodorants, for example at a concentration of up to about 10% w/w. Suitable deodorant actives can comprise deodorant effective concentrations of antiperspirant metal salts, deoperfumes, and/or microbicides, including particularly bactericides, such as chlorinated aromatics, including biguanide derivatives, of which materials known as triclosan (Irgasan DP300™), chlorhexidine and Tricloban™, warrant specific mention. A yet another class comprises biguanide salts such as available under the trade mark Cosmosil™.

A yet further class of antimicrobial which can advantageously be employed herein comprises transition metal chelators, such as amino acids or salts thereof, which chelators have affinity for iron (III), and preferably a binding constant for iron (III) of greater than 10¹⁰, or, for optimum performance, greater than ₁₀²⁶. The ‘iron (III) binding constant’ referred to above is the absolute stability constant for the chelator-iron (III) complex. One especially preferred chelator is DTPA (diethylene triamine pentaacetic acid) and salts thereof. Such antimicrobials suppress microbial regrowth. A convenient amount is from 0.35 to 2% by weight.

In practice, an optional though highly desirable component comprises a wash-off aid, preferably at a concentration of from at least 0.2% to 10% by weight and particularly from 0.5% to 5% by weight of the formulation. It assists the removal of the formulation from the skin to control build-up on the skin. The wash-off aid is and particularly at least 1%, such as up to 5% w/w of the formulation. Expressed in alternative fashion, the wash-off aid is desirably present in a weight ratio to the water-immiscible oil of from 1:10 to !:100, and especially from 1:5 to 1:40 w/w. The wash off aid is commonly a non-ionic surfactant, often having an HLB value of from about 6 to about 15, and especially is a polyalkylene oxide (eg PEO or PEO/PPO) ether or ester derivative of a fatty alcohol or acid, possibly including an intermediary polyhydric alcohol residue, eg from glycerol. Examples include seteth-15, steareth-25 and ceteareth-20.

Other optional ingredients can be incorporated to the extent that they are miscible with the carrier fluids. They include skin benefit agents such as glycerol as mentioned previously herein, and allantoin or lipids, for example in an amount of up to 5%; oil-soluble colorants; skin cooling agents such as menthol and menthol derivatives, often in an amount of up to 2%, all of these percentages being by weight of the formulation. A commonly employed and highly desired ingredient is a perfume, which is normally present at a concentration of from 0 to 4% and in many formulations from 0.25 to 2% by weight thereof.

The formulations described herein can be produced by any method that has been described previously for preparing an anhydrous soft solid formulation in which a particulate antiperspirant active is suspended in a water-immiscible oil that is structured into a solid mass by incorporation of a structurant and a thickening polymer, the conditions selected taking into account the temperature at which the various structurant(s) and thickening polymer melt or form an homogeneous blend with the carrier fluid(s).

In general, the preparative process comprises introducing the alditol into the water-immiscible oil or blend of oils and both heating and agitating the resultant mass until the alditol dissolves in the oil forming an homogenous blend. This is commonly in the region of above 115° C. up to about 140° C. Thereafter, the blend is allowed to cool or cooled to below 115° C., such as from 95 to 115° C., and any co-structurant is introduced with mixing and heating until it has dissolved. Thereafter, the mixture continues to cool or be cooled to the region of 90 to 95° C. and the polymeric thickener is introduced with stirring and the temperature maintained until the mixture is once again homogeneous.

In a separate step, the antiperspirant actives are introduced into the formulation. Although this can be before the mixture of oil and structurant(s) and thickener is obtained, it occurs preferably after the mixture containing all the structurants and the thickener has been homogenised, and in many instances can occur after the mass has cooled somewhat. A temperature range of from 85 to 90° C. is often considered acceptable.

The further ingredients of the formulation are introduced at a time of convenience to the producer. Thus, for example, it can be particularly convenient to introduce particulate materials together with the antiperspirant active, and wash-off aids into the oil together with the structurant, co-structurant or thickener. Any temperature sensitive ingredients, of which perfume can be one, are most preferably added last, and at the lowest temperature, which may even be lower than that at which the antiperspirant active is added.

When the formulation has been produced in a fluid form, it is then packaged. This is commonly achieved by shear mixing and allowing cooling of the mixture to a temperature slightly higher than its setting temperature, such as 5 to 10° C. higher, introducing the fluid mixture into a dispensing container, and thereafter cooled or allowed to cool to below the setting temperature of the formulation. The setting temperature of the formulation is determined in a prior test on a small scale, and for the invention formulations commonly resides in the range of from 60 to 80° C., depending on the selection of the constituents, their relative amounts and amongst other parameters on how much temperature moderator is employed. In order to encourage the formulation to adopt a soft solid form rather than a firm solid form, and in a variation to the filling process indicated above, the formulation is subjected to high shear mixing at or through the temperature at which the formulation would normally solidify, i.e. in the absence of shearing. Alternatively, fluidity can be attained by injecting the composition under pressure into the dispenser. The formulations herein are capable of being dispensed using soft solid dispensers such as those described in U.S. Pat. No. 5,000,356, U.S. Pat. No. 5,639,622, U.S. Pat. No. 5,725,133, or U.S. Pat. No. 6,039,483. The dispenser commonly contains from 10 to 100 g formulation, such as from 40 to 55 g.

The invention formulations can be applied to skin in the conventional manner by extruding a desired amount of formulation onto the contact surface of the head of the dispensing container, normally through one or more apertures in the head, and thereafter wiping the head across the surface of the skin, and particularly in the axilla.

Having given a detailed description of and preferences for the invention above, certain embodiments thereof will now be described more fully by way of example.

The formulations described in Examples 1 to 3 and summarised in Tables 1 to 3 were prepared by the following general method.

A solution of the structurant, i.e. the di-benzylidene alditol or substituted dibenzylidene alditol, in the carrier fluid or mixture of carrier fluids was made by heating a mixture of the carrier and structurant with agitation to and maintaining it at a temperature sufficiently high that the structurant dissolved. This was approximately 140° C. or down to about 125° C. in the presence of a temperature moderator. The solution continued to be agitated and was allowed to cool to between 95 and 115° C. and the co-structurant (where employed) was added and the agitation continued until it dissolved. The mixture was then allowed to cool to 90-95° C. when the polymeric thickener was introduced. When an homogenous mixture was obtained, it was then allowed to cool to 85-90° C., whereupon the particulate aluminium antiperspirant active was added and agitated. The mixture was next allowed to cool with continued agitation to 5-10° C. above its gelling temperature (determined in a preliminary experiment) and introduced into dispensing containers for soft solids. These were then permitted to cool to room temperature.

The following properties of the resultant soft solid formulations were determined:—

i) Hardness

Texture Analyser

This test apparatus can move a blunt probe into or out from a sample at a controlled speed and at the same time measure the applied force. The parameter which is determined as hardness is a function of the force and the projected area of indentation.

A specific test protocol used a Stable Micro systems TA.XT2I™ Texture Analyser. A sample of composition was made by heating the ingredients, pouring into a container and allowing to cool as described above. The container was a 15 ml glass jar with a wide mouth. A metal sphere, of diameter 9.5 mm, was attached to the underside of the Texture Analyser's 5 kg load cell such that it could be used for indenting a sample placed beneath it on the base plate of the instrument. After positioning the sample, the sphere position was adjusted until it was just above the sample surface. Texture Expert Exceed™ software was used to generate the subsequent motion profile used in the test method. This profile initially moved the sphere into contact with the sample and then indented the sphere into the sample at an indentation speed of 0.05 mm/s for a distance of 7 mm. At this distance the direction of motion of the sphere was immediately reversed to withdraw the sphere from the sample at the same speed of 0.05 mm/s. During the course of the test, the data acquired were time(s), distance (mm) and force (N) and the data acquisition rate was 25 Hz.

The data associated with each test were manipulated using standard spreadsheet software and used to calculate the hardness, H, at a travelled distance of 4.76 mm after initial contact with the sample, using the following equation:

H=F/A

(H expressed in N.mm⁻², F in N and A in mm⁻²

where F is the load at the same travelled distance and A is the projected area of the indentation. This area can be calculated geometrically and is equal to the area of a diametral plane of the sphere, i.e. Π×(4.76)² mm².

ii) Whiteness of Deposit

Another test of the properties of a composition is the whiteness and hence opacity of the composition which is delivered onto a surface when the composition is drawn across that surface (representing the application of the composition to human skin). To carry out this test of deposition, a sample of the composition was first applied to a test fabric under standardised conditions.

The test fabric was a rectangular strip of black worsted wool fabric 9 cm by 15 cm. This was placed in an apparatus consisting of a metallic base onto which was hinged a metallic frame defining a rectangular aperture of 5 cm by 9 cm. The test portion of fabric was laid on the base. The hinged frame was placed over the fabric and secured to the base by means of two screws thereby clamping the test fabric in place but exposing an area of 5×9 cm through the aperture.

A sample of soft solid composition in a dispensing container was kept at ambient laboratory temperature (about 20° C.) before it was required for measurement. A portion of the composition is then extruded from the container through the dispensing apertures at one end. A weight amount (0.5 g) of the extruded composition was spread uniformly across the 5×9 cm area of test fabric enclosed by the frame. Spreading was carried out using a plastic spreading tool. After spreading the sample of composition on the fabric substrate, it was removed from the apparatus and weighed to check that the mass of applied sample was 0.5±0.01 g.

The fabric with applied sample of composition was then assessed twice for whiteness, once after one hour and again after 24 hours.

This measurement was carried out using a Sony XC77™ monochrome video camera with a Cosmicar™ 16 mm focal length lens positioned vertically above a black table illuminated from a high angle using fluorescent tubes to remove shadowing. The apparatus was initially calibrated using a reference white card, after the fluorescent tubes had been turned on for long enough to give a steady light output. The cloth with a deposit thereon was placed on the table and the camera was used to capture an image. An area of the image of the deposit was selected and analysed using a Kontron IBAS™ image analyser. This notionally divided the image into a large array of pixels and measured the grey level of each pixel on a scale of 0 (black) to 255 (white). The average of the grey intensity was calculated. This was a measure of the whiteness of the deposit, with higher numbers indicating a whiter deposit. It was assumed that low numbers show a clear deposit allowing the substrate colour to be seen. All samples were prepared in triplicate and a mean of the three measured values was reported.

iii) Light Transmission

The translucency of a composition may be measured by placing a sample of standardised thickness in the light path of a spectrophotometer and measuring transmittance, as a percentage of light transmitted in the absence of the gel.

We have carried out this test using a dual-beam spectrophotometer. The sample of composition was poured hot into a 4.5 ml cuvette made of polymethylmethacrylate (PMMA) and allowed to cool to an ambient temperature of 20-25° C. Such a cuvette gives a 1 cm thickness of composition.

Measurement was carried out at 580 nm, with an identical but empty cuvette in the reference beam of the spectrophotometer, after the sample in the cuvette had been held for 24 hours. We have observed that a composition which gives a transmittance of only a fraction of 1% in this test is perceived by eye as “translucent” when extruded in a thickness less than 0.5 cm. A transmittance measured at any temperature in the range from 20-25° C. is usually adequately accurate, but measurement is made at 22° C. if more precision is required.

The above test procedures were applied to two soft solid compositions currently marketed commercially, neither of which is translucent when extruded through a narrow orifice in the dispenser. An existing product structured with castor wax and a silicone wax had a hardness by texture analyser of 0.0231 N/mm², a whiteness measurement after 1 hour of 23 and a whiteness measurement after 24 hours of 42. A competitor's product, believed also to have a glyceride wax structuring system had a hardness of 0.0318 N/mm², a whiteness measurement after 1 hour of 20, a whiteness measurement after 24 hours of 83, and a % transmittance of 0.004.

The constituents employed in the Examples were as follows:—

1) Dibenzylidene Sorbitol, (DBS) Disorbene LC™ from Roquette

2) Rheopearl KL™ from Chiba Flour Milling Co.

3) GP-1™ from Ajinomoto

4) Finsolv TN™ from Finetex

5) Propylene carbonate from Aldrich

6) Eutanol G™ from Cognis

7) Milled activated aluminium chlorohydrate from Summit (water content 17.2%)

8) Milled activated aluminium chlorohydrate from Summit (water content 7.7%)

9) Antaron WP™ 660 from ISP

10) Al/Zr tetrachlorohydrex glycine complex from Summit

11) Trans-(1R,2R)-di-dodecanamido cyclohexane (own preparation K7 in Ex 1 of GB patent application no 0019231.0)

12) Milled activated aluminium chlorohydrate from Giulini (water content 8.9%)

13) Dibenzylidene Xylitol, (DBX) experimental grade material from Roquette

14) Di(meta-flouro benzylidene) sorbitol, (mFDBS) experimental grade material from Roquette

15) Di(meta-methoxy benzylidene) sorbitol, (mMDBS) experimental grade material from Roquette

### EXAMPLE 1

In this Example, various soft solids were made that were translucent when extruded through narrow apertures.

TABLE 1

| | Example No | | | | |
|---|---|---|---|---|---|
| | 1.1 | 1.2 | 1.3 | 1.4 | 1.5 |
| Ingredients | Proportion by Weight | | | | |
| DBS (1) | 0.3 | 0.3 | 0.25 | 0.25 | 0.2 |
| Dextrin Palmitate (2) | 5 | 5 | 3 | 2 | 3 |
| N-Lauroyl-glutamic acid | | | 0.5 | 0.5 | 0.6 |
| di-n-butylamide (3) | | | | | |
| C12-15 alkyl benzoate | 65.74 | 55.4 | 67.92 | 68.88 | 67.87 |
| (4) | | | | | |
| propylene carbonate (5) | 3.46 | | 2.83 | 2.87 | 2.83 |
| octyldodecanol (6) | | 13.8 | | | |
| AACH A418 (7) | 25.5 | 25.5 | 25.5 | 25.5 | 25.5 |

Properties

| RI Active | 1.512 | 1.512 | 1.512 | 1.512 | 1.512 |
|---|---|---|---|---|---|
| RI Solvent | 1.481 | 1.478 | 1.482 | 1.482 | 1.482 |
| % transmittance | 0.14 | 0.12 | 0.13 | 0.14 | 0.16 |
| Hardness (N/mm²) | 0.018 | 0.009 | 0.012 | 0.005 | 0.012 |
| Visible Deposits (1 hr) | 11 | n/d | 11 | n/d | 11 |
| Visible Deposits (24 hr) | 10 | n/d | 10 | n/d | 10 |
| | Example No | | | | |
| | 1.6 | 1.7 | 1.8 | 1.9 | 1.10 |
| Ingredients | Proportion by Weight | | | | |
| DBS (1) | 0.2 | 0.25 | 0.2 | 0.35 | 0.2 |
| Dextrin Palmitate (2) | 4 | 3 | 5 | 5 | |
| N-Lauroyl-glutamic acid | 0.4 | 0.5 | 0.6 | | |
| di-n-butylamide (3) | | | | | |
| Triacontenyl vinyl | | | 5 | | |
| pyrrolidone | | | | | |
| copolymer (9) | | | | | |
| C12-15 alkyl benzoate | 67.1 | 65.8 | 65.3 | 69.15 | 69.3 |
| (4) | | | | | |
| propylene carbonate (5) | 2.8 | 4.95 | 3.4 | | |
| AACH A418 (7) | 25.5 | 25.5 | | 25.5 | 25.5 |
| AACH A418 (8) | | | 25.5 | | |

Properties

| | | | | | |
|---|---|---|---|---|---|
| RI Active | 1.512 | 1.512 | 1.538 | 1.512 | 1.512 |
| RI Solvent | 1.482 | 1.480 | 1.481 | 1.484 | 1.484 |
| % transmittance | 0.20 | 0.13 | 0.06 | 0.18 | 0.13 |
| Hardness (N/mm²) | 0.024 | 0.013 | 0.007 | 0.014 | 0.008 |
| Visible Deposits (1 hr) | 8 | 11 | n/d | n/d | n/d |
| Visible Deposits (24 hr) | 7 | 11 | n/d | n/d | n/d |

All formulations in this Example left no visible deposits on skin when applied topically, nor when left on.

### EXAMPLE 2

Further soft solid formulations using alternative alditols were made that were translucent when extruded through narrow apertures.

TABLE 2

| | Example No | | | |
|---|---|---|---|---|
| | 2.1 | 2.2 | 2.3 | 2.4 |
| | Proportion by weight | | | |
| Ingredients | | | | |
| DBS (1) | | | | 0.2 |
| DBX (13) | 0.4 | | | |
| m-flouro DBS (14) | | | 0.2 | |
| m-methoxy DBS (15) | | 0.2 | | |
| Dextrin Palmitate (2) | 5.0 | 4.0 | 4.0 | 4.0 |
| N-Lauroyl-glutamic acid di- | | 0.4 | 0.4 | |
| n-butylamide (3) | | | | |
| K7 (11) | | | | 0.4 |
| C12-15 alkyl benzoate (4) | 69.1 | 67.1 | 67.1 | 67.1 |
| propylene carbonate (5) | | 2.8 | 2.8 | 2.8 |
| AACH A418 (7) | 25.5 | 25.5 | | |
| Aloxicoll LR (12) | | | 25.5 | 25.5 |

Properties

| | | | | |
|---|---|---|---|---|
| RI Active | 1.512 | 1.512 | 1.530 | 1.530 |
| RI Solvent | 1.484 | 1.482 | 1.482 | 1.482 |
| % transmittance | 0.2 | 0.21 | 0.07 | 0.07 |
| Hardness (N/mm²) | 0.018 | 0.018 | 0.030 | 0.014 |
| Visible Deposits (1 hr) | 11 | n/d | n/d | n/d |
| Visible Deposits (24 hr) | 11 | n/d | n/d | n/d |

All formulations in this Example left no visible deposits on skin when applied topically, nor when left on.

### EXAMPLE 3

In this Example, a formulation was produced which was opaque when extruded through a narrow aperture.

TABLE 3

| | Example No |
|---|---|
| | 3.1 |
| | % w/w |
| Ingredients | |
| DBS (1) | 0.2 |
| Dextrin Palmitate (2) | 4 |
| N-Lauroyl-glutamic acid di- | 0.4 |
| n-butylamide (3) | |
| C12-15 alkyl benzoate (4) | 67.1 |
| propylene carbonate (5) | 2.8 |
| AZAG 7167 (10) | 25.5 |
| Properties | |
| RI Active | 1.562 |
| RI Solvent | 1.482 |
| % Transmittance | 0.002 |
| Hardness (N/mm²) | 0.009 |
| Visible Deposits (1 hr) | 11 |
| Visible Deposits (24 hr) | 11 |

Although opaque when extruded onto the head of the dispenser, the formulation of this Example could be applied easily to the skin and left no visible deposits.

1 An anhydrous antiperspirant formulation in the form of a soft solid comprising:
a particulate antiperspirant salt in an amount of from 5 to 30% by weight;
an anhydrous carrier fluid in an amount of from 50 to 85% by weight in which at least 70% by weight of the carrier fluid is selected from branched fatty alcohols; aliphatic esters and aromatic esters, and
a structurant system for the anhydrous carrier fluid comprising:
i) a dibenzylidene alditol in an amount of from 0.05 to 1.5% by weight and
ii) a polymeric thickener in an amount of at least 2% by weight.

2 A composition according to claim 1 in which the dibenzylidene alditol is dibenzylidene sorbitol, a halo or methoxy substituted dibenzylidene sorbitol or dibenzylidene xylitol.

3 A composition according to claim 1 in which the dibenzylidene alditol is employed in an amount of from 0.1 to 0.5% by weight.

4 A composition according to claim 1 additionally employing a temperature moderator.

5 A composition according to claim 4 in which the temperature moderator is propylene carbonate.

6 A composition according to claim 4 in which the temperature moderator is employed in a weight ratio to the dibenzylidene alditol of up to 20:1 and preferably from 8:1 to 14:1.

7 A composition according to claim 1 in which the polymeric thickener comprises a dextrin fatty acid ester.

8 A composition according to claim 7 in which the dextrin fatty acid ester satisfies the formula 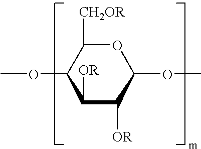
in which each R group, individually, represents a hydrogen atom or an acyl group having up to 22 carbon atoms, provided that at least one R group per glucose unit is an acyl group of at least 4 carbon atoms, and m has an average value from 5 to 50.

9 A composition according to claim 8 in which the number of glucose units is from 20 to 30.

10 A composition according to claim 9 in which the polymeric thickener is employed in a weight ratio to the dibenzylidene alditol of from 5:1 to 30:1, and preferably from 8:1 to 20:1.

11 A composition according to claim 1 in which the amount of polymeric thickener is up to 7%.

12 A composition according to claim 1 which further contains a fibre-forming co-structurant.

13 A composition according to claim 12 in which the co-structurant is selected from
hydroxystearic acids and derivatives thereof structurants which include an amido group;
combinations of a sterol and a sterol ester;
and structurants satisfying general formula 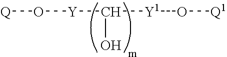
in which in which Y and Y¹ each represents a methylene or carbonyl group, m is 1 or 2 and Q and Q1 each represent an aralkylene group.

14 A composition according to claim 13 in which the co-structurant is an N-acylamino acid amide, preferably N-lauryl-L-glutamic acid di-n-butylamide.

15 A composition according to claim 13 in which the co-structurant is an amido-containing material having a general formula 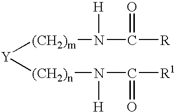
in which Y represents a cyclohexane ring bearing the two amido substituents in the formula in a 1, 2 spacial relationship, m and n are each 0 and R and R¹ are the same and each represents an alkyl group containing from 11 to 17 carbons.

16 A composition according to claim 12 in which the co-structurant is employed in an amount of up to 2% by weight.

17 A composition according to claim 12 in which the weight ratio of polymeric thickener to co-structurant is in the range of from 3:1 to 12:1.

18 A composition according to claim 1 in which the antiperspirant active is an astringent aluminium or aluminium/zirconium salt or complex.

19 A composition according to claim 18 in which the antiperspirant active is an aluminium chlorohydrate, activated aluminium chlorohydrate or a glycine complex of an aluminium/zirconium chlorohydrate.

20 A composition according to claim 19 in which the antiperspirant active comprises void-free or milled particles.

21. A composition according to claim 1 in which the carrier fluid is selected from branched fatty alcohols, aliphatic esters and aromatic esters having a melting point of below 25° C.

22 A composition according to claim 21 in which the carrier fluid comprises an alkyl benzoate.

23 A composition according to claim 21 in which the weight ratio of alcohol to ester or esters is from 1:1 to 10:1.

24 A composition according to claim 20 in which the composition contains one or more co-carrier fluids selected from hydrocarbon oils, polyoxyalkylene oxide alkyl ethers and silicone fluids.

25 A composition according to claim 24 in which said carrier fluids constitute at least 80% and preferably at least 90% of the total proportion of carrier fluids.

26 A composition according to claim 24 in which the proportion of silicone oils of the carrier fluids is not more than 5% by weight.

27 A process for making an anhydrous antiperspirant soft solid composition comprising the steps of:
i) introducing into a mixing vessel a water-immiscible carrier fluid in an amount of from 50 to 85% by weight in which at least 70% by weight of the carrier fluid is selected from branched fatty alcohols, aliphatic esters and aromatic esters;
ii) introducing into the vessel structurant system comprising a dibenzylidene aiditol in an amount of at from 0.05 to 1.5% by weight and a polymeric thickener in an amount of least 2% by weight;
iii) heating the structurant system until it melts or is miscible with the carrier fluid;
iv) introducing into the carrier fluid or mixture of carrier fluid and structurant a particulate aluminium/zirconium astringent salt in an amount of from 5 to 30% by weight at a temperature above the normal solidification temperature of the formulation and;
v) introducing the composition into a dispenser whilst the composition is fluid.

28 A cosmetic method for controlling or reducing perspiration in which a composition according to claim 1 is applied topically to human skin.
